(19) United States
(12) Patent Application Publication
(10) Pub. No.: US 2022/0265641 A1
(43) Pub. Date: Aug. 25, 2022

(54) SEROTONERGIC AGENT AND 5-HT1A-RECEPTOR ANTAGONIST

(71) Applicant: ATLAS PHARMACEUTICALS BV, BRUGGE (BE)

(72) Inventors: Marcel David Waldinger, AMSTEL VEEN (NL); Berend Olivier, LELYSTAD (NL)

(21) Appl. No.: 17/627,602

(22) PCT Filed: Jul. 29, 2020
(86) PCT No.: PCT/EP2020/071410

§ 371 Date: Jan. 14, 2022

(30) Foreign Application Priority Data
Jul. 29, 2019 (NL) 2023581

(51) Int. Cl. A61K 31/496; A61P 15/12

(52) U.S. Cl. CPC A61K 31/496; A61P 15/12

(57) ABSTRACT

The present disclosure relates to a 5-HT1A-receptor antagonist or a derivative, precursor or metabolite thereof, in combination with at least one serotonergic agent or a derivative, precursor or metabolite thereof for use in the prevention and/or treatment of premature ejaculation, wherein the 5-HT1A-receptor antagonist or a derivative, precursor or metabolite thereof is administered separately, sequentially or simultaneously to the at least one serotonergic agent or a derivative, precursor or metabolite thereof.

### CROSS-REFERENCE TO RELATED APPLICATIONS

This application is a national phase entry under 35 U.S.C. § 371 of International Patent Application PCT/EP2020/071410, filed Jul. 29, 2020, designating the United States of America and published as International Patent Publication WO 2021/018967 A1 on Feb. 4, 2021, which claims the benefit under Article 8 of the Patent Cooperation Treaty to Dutch Patent Application Serial No. 2023581, filed Jul. 29, 2019.

### TECHNICAL FIELD

This application relates generally to medicine and more particularly to treatment of Premature Ejaculation (PE).

### BACKGROUND

Selective serotonin reuptake inhibitors (SSRIs) have been previously suggested for the treatment of Premature Ejaculation (PE). Before the effects on PE emerge, several weeks of chronic treatment are necessary. Ideally, a medication should be used that can be taken shortly before sexual intercourse and that works on demand (i.e., immediately or within hours).

Although dapoxetine (an SSRI) has been introduced for ‘on demand’ use in PE, the existing data does not support ‘on demand’ properties of dapoxetine. This seems supported by the high discontinuation rate of the product, which may be caused by undesirable side effects of dapoxetine.

### BRIEF SUMMARY

The disclosure provides for the use of at least one serotonergic agent, or a derivative, precursor or metabolite thereof, in combination with at least one 5-HT1A-receptor antagonist, or a derivative, precursor or metabolite thereof, in the prevention and/or treatment of premature ejaculation (PE), wherein the at least one serotonergic agent or a precursor or metabolite thereof is administered separately, sequentially or simultaneously to the at least one 5-HT1A-receptor antagonist. Preferably, the at least one serotonergic agent, or a precursor or metabolite thereof is at least one SSRI and the at least one 5-HT1A-receptor antagonist is DU125530.

The combined administration of a serotonergic agent and a 5-HT1A-receptor antagonist, surprisingly, was found to increase the latency time to (first) ejaculation with fast onset of action, thus providing for an ‘on demand’ medication for premature ejaculation.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one.”

### BRIEF DESCRIPTION OF THE DRAWINGS

FIG. 1 are two graphs showing the effects of treatments with vehicle (Vh+Vh), the SSRI paroxetine (5-mg/kg: Px+Vh), the combination of the 5-HT1A-receptor antagonist WAY100,635 (0.3-mg/kg) and paroxetine (5-mg/kg)(WAYY+Px) and the combination of DU125530 (20-mg/kg) and paroxetine (5-mg/kg)(Du+Px).

FIG. 2 is a schematic depicting the synthesis of DU125530.

FIG. 3 is a graph depicting an expected dose-response curve of combinations of the 5-HT1A receptor antagonist DU125530 and an SSRI on the ejaculation latency (seconds) of sexually trained rats in a 30-minute meeting with a female rat in estrus.

### DETAILED DESCRIPTION

The disclosure relates to at least one serotonergic agent, or a derivative, precursor or metabolite thereof, in combination with at least one 5-HT1A-antagonist, or a derivative, precursor or metabolite thereof, for use in a method for delaying the ejaculation latency time, in particular, for prevention and/or treatment of premature ejaculation (PE), wherein the at least one serotonergic agent or a precursor or metabolite thereof is administered separately, sequentially or simultaneously to the at least one 5-HT1A-antagonist to a subject in need of thereof. Preferably, the at least one serotonergic agent is at least one SSRI and the at least one 5-HT1A-receptor antagonist is DU125530.

Premature ejaculation (PE), also referred to as early ejaculation or ejaculation praecox, occurs when a male subject experiences orgasm and expels semen relatively soon after initiating sexual activity and with relatively little stimulation. In the context of the disclosure, “premature” is defined as at most one minute after penetration (in accordance with i.a. Serefoglu et al; Sexual medicine. 2 (2): 41-59) or within 15 seconds from the beginning of sexual intercourse (in accordance with The International Classification of Diseases (ICD-10)).

Alternatively and/or additionally, Premature ejaculation, under evidence-based criteria generated by the International Society for Sexual Medicine in 2014, can be defined as being not the result of a nonsexual mental illness, a problem in a given relationship or caused by medication, by the person ejaculating within one minute after penetration and before the person wants to ejaculate, occurring for a duration longer than 6 months and happening (almost) every time (or in 90% of cases), and causing significant distress for person. These factors can be identified by talking with the male subject, not through any diagnostic test. Premature ejaculation (PE) can also be defined as ejaculating without control, and/or within at most 15 seconds or at most 1, 2, or 3 minutes after initiating penetration (according to the 2007 ICD-10).

In a preferred embodiment, the disclosure aims for increasing (latency) time to ejaculation in (human) males.

In the context of the disclosure, a serotonergic agent is a compound that modifies the effects of serotonin in the body and/or a compound that produces its effects via interactions with the serotonergic system, for example, by stimulating or blocking serotonin neurotransmission. Different types of serotonergic agents may be used, including the following:

Serotonin reuptake inhibitors;

Serotonin-norepinephrine reuptake inhibitors (SNRIs);

Serotonin receptor agonist or antagonist;

Serotonin releasing agents; and/or

Any agent that has an SSRI-component in its working mechanism (e.g., tramadol).

In the context of the disclosure, an SSRI is a compound that modifies the effects of serotonin in the body and/or a compound that produces effects of serotonin via interactions with the serotonergic system, by stimulating serotonin neurotransmission.

In the disclosure, a serotonin reuptake inhibitor (SRI) is preferred. An SRI is a compound that acts as a reuptake inhibitor of the neurotransmitter serotonin (5-hydroxytryptamine (5-HT)) by e.g., blocking the action of the serotonin transporter (SERT). This in turn leads to increased extracellular concentrations of serotonin and, hence, an increase in serotonergic neurotransmission.

Particularly preferred are selective serotonin reuptake inhibitors (SSRIs). SSRIs are a class of compounds that are also used as antidepressants in the treatment of (major) depressive disorder and anxiety disorders. The exact mechanism of action of SSRIs in depression, anxiety and ejaculation is unknown. SSRIs are believed to increase the extracellular level of the neurotransmitter serotonin by limiting its reabsorption (reuptake) into the presynaptic cell, increasing the level of serotonin in the synaptic cleft available to bind to postsynaptic receptors. They may have varying degrees of selectivity for other monoamine transporters, with pure SSRIs having only weak affinity for the norepinephrine and dopamine transporters. Suitable Examples of SSRIs include Citalopram, Escitalopram, Fluoxetine, Fluvoxamine, Paroxetine, Sertraline, Dapoxetine, Indalpine, Zimelidine, Alaproclate, Centpropazine, Cericlamine (JO-1017), Femoxetine (Malexil®; FG-4963), Ifoxetine (CGP-15210), Omiloxetine, Panuramine (WY-26002), Pirandamine (AY-23713), Seproxetine ((S)-norfluoxetine).

SNRIs inhibit the reuptake of serotonin and norepinephrine. Suitable examples include Atomoxetine, Desvenlafaxine, Duloxetine, Levomilnacipran, Milnacipran, Sibutramine, Tramadol, and/or Venlafaxine.

The serotonergic agent according to the present disclosure may also be Chlorimipramine.

The serotonergic agent, or a derivative, precursor or metabolite thereof, is preferably given in a formulation wherein there is a short-lasting high peak of serotonergic agent in the blood circulation, for example, in the form of an oral or sublingual formulation, for example, a tablet or a sublingual formulation with cyclodextrins as carrier. Preferably, the serotonergic agent is an SSRI. Another example of a suitable route of administration of serotonergic agent is buco-, mucosally, or intranasally. The dose of serotonergic agent, or a derivative, precursor or metabolite thereof, in the formulation may be between 0.1-10 mg, preferably between 2-8 mg. The present disclosure aims at a temporary increase in the serotonergic agent blood plasma level in the treated subject (short lasting peak). The term “short-lasting” refers to an application of serotonergic agent such that the serotonergic agent levels in the blood plasma are back to base-line level within 2-4, or 3-5 hours after administration.

As described above, the disclosure combines at least one serotonergic agent, or a derivative, precursor or metabolite thereof, with at least one 5-HT1A-receptor antagonist, or a derivative, precursor or metabolite thereof. Preferably, the at least one serotonergic agent is at least one SSRI and the at least one 5-HT1A-receptor antagonist is DU125530.

The 5-HT1A receptor (or serotonin 1A receptor) is a subtype of serotonin receptors (5-HT receptors) that binds the neurotransmitter serotonin (5-hydroxytryptamine, 5-HT). It is a G protein-coupled receptor (GPCR), coupled to the Gi protein that mediates inhibitory neurotransmission. The 5-HT1A receptor protein is encoded by the HTR1A gene. A 5-HT1A-receptor antagonist is a type of receptor ligand or compound that blocks or inhibits a biological response by binding to and/or blocking the 5-HT1A receptor rather than activating it like an agonist. Suitable examples of 5-HT1A-receptor antagonists include Alprenolol, AV-965, BMY-7,378, Cyanopindolol, Cyproheptadine, Dotarizine, Flopropione, GR-46,611, Iodocyanopindolol, Isamoltane, Lecozotan, Mefway, Methiothepin, MPPF, NAD-299, NAN-190, Nebivolol, Oxprenolol, Pindobind, Pindolol, Propranolol, Risperidone, Robalzotan, SB-649,915, SDZ-216,525, Spiperone, Spiramide, Spiroxatrine, UH-301, WAY100,635 and WAY-100,135.

A majority of the above mentioned 5-HT1A receptor antagonists possess additional pharmacological mechanisms next to 5-HT1A receptor antagonism, often showing disturbing side effects. 5-HT1A-receptor antagonists may stimulate or block serotonergic non-5-HT1A receptors and/or non-serotonergic receptors, may have biased agonistic effects at the 5-HT1A receptor, or may have differential antagonistic/agonistic effects at pre- or postsynaptically located 5-HT1A receptors. Moreover, some 5-HT1A receptor antagonists have partial 5-HT1A receptor agonistic effects, are racemic mixtures of antagonistic/agonistic enantiomers, which may lead to unwanted side effects.

Many 5-HT1A receptor antagonists are primarily, because of another mechanism than 5-HT1A antagonistic effects, used for other purposes, e.g., for heart disease. Such 5-HT1A receptor antagonists can be, for example, beta-blockers, that potentially lead to lowered or irregular heart rate and/or lowered blood pressure. Several 5-HT1A receptor antagonists are used for certain psychiatric disorders such as schizophrenia and have dopaminergic mechanisms, leading to unwanted dopaminergic side effects. 5-HT1A receptor antagonists may have low penetration of the blood brain barrier, decreasing their effectiveness.

The 5-HT1A receptor antagonist according to the disclosure is preferably characterized by Formula I:

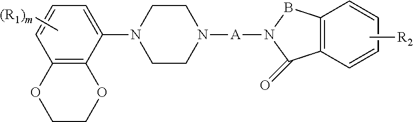

wherein
R₁ is a halogen, methyl, methoxy, hydroxyl, trifluoromethyl or cyano, preferably chlorine;
m is 1 or 2, preferably 1;
R₂ is hydrogen or chlorine, preferably hydrogen;
A represents an alkylene chain containing 2-6 C-atoms, preferably containing 3 C-atoms, which may be substituted with a phenyl group, and;
B is methylene, ethylene, carbonyl, sulfinyl, sulfonyl or sulfur, preferably sulfonyl.

Compounds of Formula I or salt thereof in combination with at least one serotonergic agent (e.g., SSRI) or salt thereof as according to the present disclosure can be used in the prevention and/or treatment of premature ejaculation, wherein the compound of Formula I is preferably administered separately, sequentially or simultaneously to the at least one serotonergic agent.

Surprisingly, the combination of a compounds of Formula I and at least one serotonergic agent (preferably, an SSRI) as according to the present disclosure markedly increase ejaculation latency time.

A particularly preferred 5-HT1A-receptor antagonist is DU125530. DU125530 in combination with at least one SSRI further increases ejaculation latency time. In addition, the technical effect of further increasing ejaculation latency time with the combination of DU125530 and at least one SSRI, can be obtained with a lowered dose of SSRI, comparable to a dose of SSRI required to treat depression. A lowered dose of SSRI is associated with reduced occurrence of side effects and symptoms of these side effects being less severe.

DU125530 can be characterized as a compound with Formula II

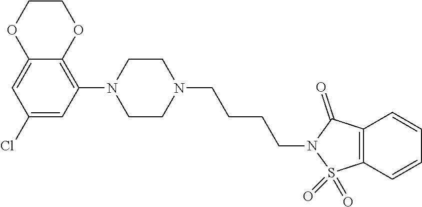 [SMILES: O=C1c2ccccc2S(=O)(=O)N1CCCCN1CCN(c2cc(Cl)cc3c2OCCO3)CC1]

(Formula II) _(2-[4-[4-(7-chloro-2,3-dihydro-1,4-benzodi-oxin-5-yl)-1-piperazinyl]butyl]-1,2-benzisothiazol-3(2H)-one-1,1-dioxide) and/or as CAS [161611-99-0], having MF C₂₃H₂₆ClN₃O₅S, and/or MW 491.99.

References to serotonergic agents and/or 5-HT1A-receptor antagonists, particularly with regard to therapeutic use, will be understood to also encompass pharmaceutically acceptable salts of thereof. Preferably, the serotonergic agent is an SSRI and the 5-HT1A-receptor antagonist is DU125530. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

The scope of the disclosure also extends to derivatives of serotonergic agents and/or 5-HT1A-receptor antagonists that retain the desired activity. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutically active agents such as, for example, improved solubility, reduced toxicity, enhanced uptake, etc. Preferably, the at least one serotonergic agent combined with the at least one 5-HT1A-receptor antagonists is formulated as a pharmaceutical composition further comprising one or more pharmaceutically acceptable carriers, excipients or diluents. Preferably, the at least one serotonergic agent is at least one SSRI and the at least one 5-HT1A-receptor antagonist is DU125530.

The route of administration for the at least one serotonergic agent, or derivative, precursor or metabolite thereof and the at least one 5-HT1A-receptor antagonist or derivative, precursor or metabolite thereof, is preferably non-invasive (for example, oral). Preferably, the at least one serotonergic agent is at least one SSRI and the at least one 5-HT1A-receptor antagonist is DU125530. Typically, administration is orally, sublingually, or by inhalation, or administered by means of a cream.

The quantity for the at least one serotonergic agent and/or the at least one 5-HT1A-receptor antagonist per unit dose may be varied according to the nature of the active compounds and the intended dosage regime. Preferably, the at least one serotonergic agent is at least one SSRI and the at least one 5-HT1A-receptor antagonist is DU125530. Generally, an effective amount shall be used, which may be within the range of from 0.01 mg to 5000 mg, preferably 0.01-4000 mg, 0.1-3000 mg, 1-2500, 1-1000, 10-100, 1-10, 1-5 mg per unit dose.

It is preferred that the different compounds as applied in the disclosure are released in the blood stream according to the following schedule:
the at least one serotonergic agent, or a derivative, precursor or metabolite thereof, is essentially released between 0.5-1.5 hours before sexual activity; and/or
the 5-HT1A antagonist, or a derivative, precursor or metabolite thereof is essentially released between 0.5-1.5 hours before sexual activity.

Preferably, the at least one serotonergic agent is at least one SSRI and the at least one 5-HT1A-receptor antagonist is DU125530.

At the same time or alternatively, dosage is such that the peak effects of the at least one serotonergic agent (or a derivative, precursor or metabolite thereof), and/or the 5-HT1A antagonist, or a derivative, precursor or metabolite thereof partly overlap. Preferably, the at least one serotonergic agent is at least one SSRI and the at least one 5-HT1A-receptor antagonist is DU125530.

Preferably, the at least one serotonergic agent or a derivative, precursor or metabolite thereof, and/or the at least one 5-HT1A-receptor antagonist or a derivative, precursor or metabolite thereof for use in the prevention and/or treatment of premature ejaculation according to the present disclosure are administered to a male subject between 24 hours and 1 minute before sexual activity, such as between 20 hours and 1 minute, 15 hours and 1 minute, 10 hours and 1 minute, 8 hours and 1 minute, 6 hours and 1 minute, 4 hours and 1 minute, 120 to 1 minutes, such as between 60 to 1 minutes, 40 to 1 minutes, 30 to 1 minutes 20 to 1 minutes, 15 to 1 minutes, 10 to 1 minutes, 5 to 1 minute, 3 to 1 minutes, wherein the serotonergic agent or a derivative, precursor, or metabolite thereof is preferably an SSRI, such as paroxetine, and wherein the 5-HT1A-receptor antagonist or a derivative, precursor, or metabolite thereof is preferably DU125530.

Preferably, the at least one serotonergic agent or a derivative, precursor, or metabolite thereof, of the at least one serotonergic agent or a derivative, precursor, or metabolite thereof, and/or the at least one 5-HT1A-receptor antagonist or a derivative, precursor, or metabolite thereof for use in the prevention and/or treatment of premature ejaculation according to the present disclosure, is administered to a male subject between 10 hours and 1 minute before administering the at least one 5-HT1A-receptor antagonist or a derivative, precursor, or metabolite thereof to the subject, such as between 8 hours and 10 minutes, between 6 hours and 30 minutes, between 5 and 1 hour, or between 4 and 2 hours before administering the at least one 5-HT1A-receptor antagonist or a derivative, precursor, or metabolite thereof. The at least one serotonergic agent or a derivative, precursor, or metabolite thereof can be administered between 4 hours and 10 minutes before sexual activity, wherein the 5-HT1A-receptor antagonist or a derivative, precursor, or metabolite thereof can be administered between 10 and 1 minute before sexual activity, wherein the serotonergic agent or a derivative, precursor, or metabolite thereof is preferably an SSRI, such as paroxetine, and wherein the 5-HT1A-receptor antagonist or a derivative, precursor, or metabolite thereof is preferably DU125530.

Preferably, the at least one 5-HT1A-receptor antagonist or a derivative, precursor, or metabolite thereof, of the at least one serotonergic agent or a derivative, precursor, or metabolite thereof, and/or the at least one 5-HT1A-receptor antagonist or a derivative, precursor, or metabolite thereof for use in the prevention and/or treatment of premature ejaculation according to the present disclosure, is administered to a male subject between 10 hours and 1 minute before administering the at least one serotonergic agent or a derivative, precursor, or metabolite thereof to the subject, such as between 8 hours and 10 minutes, between 6 hours and 30 minutes, between 5 and 1 hour, or between 4 and 2 hours before administering the at least one serotonergic agent or a derivative, precursor, or metabolite thereof. The at least one 5-HT1A-receptor antagonist or a derivative, precursor, or metabolite thereof can be administered between 4 hours and 10 minutes before sexual activity, while the serotonergic agent or a derivative, precursor, or metabolite thereof can be administered between 10 and 1 minute before sexual activity, wherein the serotonergic agent or a derivative, precursor, or metabolite thereof is preferably an SSRI, such as paroxetine, and wherein the 5-HT1A-receptor antagonist is preferably DU125530.

Preferably, the at least one serotonergic agent or a derivative, precursor, or metabolite thereof and/or the at least one 5-HT1A-receptor antagonist or a derivative, precursor, or metabolite thereof for use in the prevention and/or treatment of premature ejaculation are not for use in the prevention and/or treatment of any other condition, disorder or disease apart from premature ejaculation.

Preferably, the at least one serotonergic agent or a derivative, precursor, or metabolite thereof and/or the at least one 5-HT1A-receptor antagonist or a derivative, precursor, or metabolite thereof for use in the prevention and/or treatment of premature ejaculation according to the present disclosure are not administered periodically, such as daily, weekly, biweekly monthly, every 12 hours, every 8 hours, twice a day, three times per day, or four times per day. Preferably, wherein the serotonergic agent or a derivative, precursor, or metabolite thereof is preferably an SSRI, such as paroxetine, and wherein the 5-HT1A-receptor antagonist or a derivative, precursor, or metabolite thereof is preferably DU125530.

In a preferred embodiment, the at least one serotonergic agent (or a derivative, precursor or metabolite), and/or the at least one 5-HT1A antagonist or a derivative, precursor or metabolite as used according the present disclosure are provided as a single composition comprising the recited compounds, for example, a tablet. Preferably, the at least one serotonergic agent is at least one SSRI and the at least one 5-HT1A-receptor antagonist is DU125530.

This composition may further comprise one or more pharmaceutically acceptable carriers, excipients or diluents.

In an alternative embodiment, the at least one serotonergic agent (or a derivative, precursor or metabolite thereof), and/or the at least one 5-HT1A antagonist or a derivative, precursor or metabolite as used according the present disclosure are provided as a combination or a kit of parts comprising separate compositions, for example, a composition comprising at least one serotonergic agent (or a derivative, precursor or metabolite thereof) and/or a composition comprising at least one 5-HT1A antagonist or a derivative, precursor or metabolite. Preferably, the at least one serotonergic agent is at least one SSRI and the at least one 5-HT1A-receptor antagonist is DU125530. These separate compositions may further comprise one or more pharmaceutically acceptable carriers, excipients or diluents.

Suitable dosage forms for the at least one serotonergic agent (or a derivative, precursor or metabolite thereof), and/or the at least one 5-HT1A antagonist or a derivative, precursor or metabolite, preferably wherein the at least one serotonergic agent is at least one SSRI and the at least one 5-HT1A-receptor antagonist is DU125530, in the context of the present disclosure include, but are not limited to, solid dosage forms, for example, tablets, capsules, powders, dispersible granules, cachets and suppositories, including sustained release and delayed release formulations. Powders and tablets will generally comprise from about 5% to about 70% active ingredient. Solid carriers and excipients are generally known in the art and include, e.g., magnesium carbonate, magnesium stearate, talc, sugar, lactose, etc. Tablets, powders, cachets and capsules are all suitable dosage forms for oral administration.

Suitable liquid dosage forms for the at least one serotonergic agent (or a derivative, precursor or metabolite thereof), and/or the at least one 5-HT1A antagonist or a derivative, precursor or metabolite, preferably wherein the at least one serotonergic agent is at least one SSRI and the at least one 5-HT1A-receptor antagonist is DU125530, include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. Aerosol preparations suitable for inhalation may include solutions and solids in powder form, which may be combined with a pharmaceutically acceptable carrier, such as an inert compressed gas.

Also encompassed are dosage forms for transdermal administration for the at least one serotonergic agent (or a derivative, precursor or metabolite thereof), and/or the at least one 5-HT1A antagonist or a derivative, precursor or metabolite, for example, creams, lotions, oils, aerosols and/or emulsions. Preferably, the at least one serotonergic agent is at least one SSRI and the at least one 5-HT1A-receptor antagonist is DU125530. These dosage forms may be included in transdermal patches of the matrix or reservoir type, which are generally known in the art.

Pharmaceutical preparations dose may be conveniently prepared in unit dosage form, according to standard procedures of pharmaceutical formulation.

In the present disclosure, a “derivative” may be seen as a compound that differs from the reference compound in at least one atom (and/or chemical bond) and/or at least one functional group. The difference may also be in at least two, three, or four atoms (and/or chemical bonds) and/or at least two, three, four functional groups. In the present disclosure, the term “precursor” refers to a compound that is converted to the reference compound, for example, by the metabolism of the host. In the present disclosure, the term “metabolite” refers to a compound that results from the reference compound upon acting of the metabolism of the host.

### EXPERIMENTAL SECTION

### Example 1

A model of sexual behavior in male rats was developed that is predictive of SSRI-effects on human male sexual behavior (Bijlsma et al. 2014; Olivier et al. 2006; 2017). In this male rat sexual behavior model, SSRIs inhibit sexual behavior, but only upon chronic administration thereby mimicking and modeling the SSRI effects in human males.

Adding acutely a 5-HT1A receptor antagonist to an acute SSRI dose was found to lead to an immediate inhibition of male sexual behavior. Initially, WAY100,635, a prototypic 5-HT1A-receptor antagonist was used, but this compound is preferably not used in humans.

Adding a 5-HT1A-receptor antagonist to an SSRI was considered to possibly speed up the onset of action of the antidepressant activity. One compound, DU125530 was selected that has selective 5-HT1A receptor antagonistic activities in vitro and in vivo (Mos et al. 1997). This compound was tested in several animal models and showed a nice and reliable 5-HT1A receptor antagonistic activity, but was, given on itself without biological activity (a so-called silent antagonist) (Joordens et al. 1997). In a positron emission tomography study in healthy human volunteers (Rabiner et al. 2002), a 10 to 40-mg daily dose range was well tolerated, had minor side effects, and exhibited a dose-dependent 5-HT1A-receptor occupancy from 0-72% at 2 h post the last dose. Occupancy correlated highly with plasma levels of DU125530.

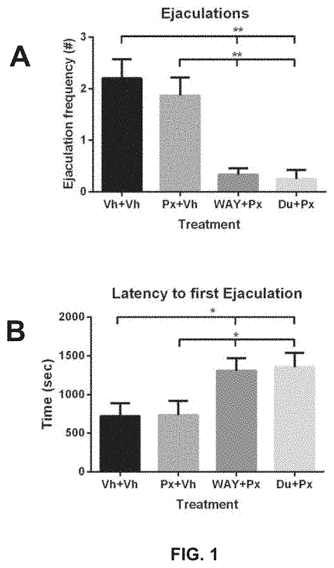

In the above-mentioned rat model, acute administration of DU125530 (20 mg/kg) in combination with acutely given paroxetine (SSRI, 5 mg/kg) strongly inhibited male rat sexual behavior. Because the prototypic 5-HT1A receptor antagonist WAY100,635 exerts a comparable inhibition (own data), it is considered that blockade of 5-HT1A receptors in combination with blockade of the serotonin transporter has acute male sexual behavior inhibiting effects, specifically on the ejaculation process (see FIG. 1).

Finding: Combination of a drug, which blocks 5-HT1A receptors (like DU125530) with a serotonergic compound such as an SSRI, leads to a fast inhibition of male rat sexual behavior, including ejaculation. This finding strongly supports the application of this combination for ‘on demand’ treatment of human premature ejaculation.

Materials and Methods

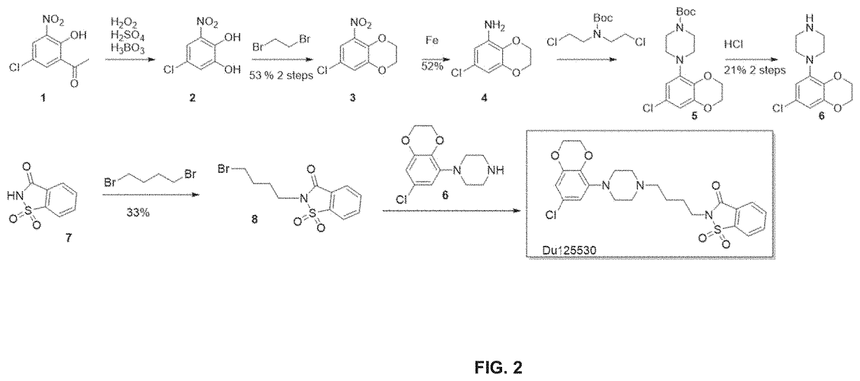

One gram of DU125530 was ordered from SYNCOM, a (medicinal) chemistry company at the Zernike Campus of RUG (University of Groningen). The synthesis route is depicted in FIG. 2 and can be reproduced by the skilled person. The compound was found safe, pharmacologically active when combined with paroxetine and had no effects when administered alone (a so-called ‘silent’ antagonist).

Male rats were sexually trained till they obtained a stable sexual performance (on average 2 ejaculations per 30-min, the standard testing duration). Animals were injected intraperitoneally 30-min before testing after which males had 30 min access to the females that were behaviorally sexually receptive. A trained researcher scored the sexual behavior of the males according to published and accepted methods (Chan et al. 2010).

Results

FIG. 1 shows the effects of treatments with vehicle (Vh+Vh), the SSRI paroxetine (5-mg/kg: Px+Vh), the combination of the 5-HT1A-receptor antagonist WAY100,635 (0.3-mg/kg) and paroxetine (5-mg/kg)(WAY+Px) and the combination of DU125530 (20-mg/kg) and paroxetine (5-mg/kg)(Du+Px). In separate experiments, the two 5-HT1A-receptor antagonists WAY100,635 (at doses up to 1 mg/kg, IP) and DU125530 (at doses up to 20-mg/kg) had no behavioral effects on sexual behavior (not shown here), illustrating that both compounds are silent antagonists.

### Example 2

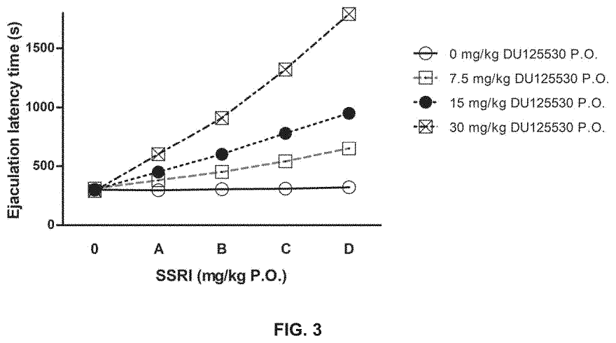

Administration of DU125530 in conjunction with an SSRI to male rats is expected to increase the ejaculation latency time of male rats. In addition, the increase of ejaculation latency time requires a lower dose of SSRI compared to SSRI administration without DU125530. The addition of DU125530 thus shifts the dose-response curve for the ejaculation latency time to the left. This is portrayed in FIG. 3, showing an expected dose-response curve of combinations of the 5-HT1A receptor antagonist DU125530 and an SSRI on the ejaculation latency (seconds) of sexually trained rats in a 30-minute meeting with a female rat in estrus. In FIG. 3, the letters A-D on the horizontal axis denote increasing SSRI concentrations and ejaculation latency time is displayed on the vertical axis. Concentrations A, B, C, and D correspond approximately with 1, 10, 20, 30 mg/kg, respectively. The abbreviation P.O. in the legend of FIG. 3 is per os, meaning oral administration. These data suggest that adding DU125530 to an SSRI will lead to lower doses of the SSRI needed to increase the ejaculation latency time. This is advantageous because at lower SSRI doses side effects of the SSRI will be lowered.

### Example 3

In a study with male rats, 10 different 5-HT1A-receptor antagonists are administered in conjunction with five different serotonergic agents shortly before sexual activity. Within each group, each male rat is administered a different serotonergic agent in conjunction with the 5-HT1A-receptor antagonist, selected from Citalopram, Fluoxetine, Ifoxetine (CGP-15210), Paroxetine, and Zimelidine, The time until first ejaculation is measured and divided by an average time until first ejaculation for the specific male rat without administration of the 5-HT1A-receptor antagonist and the serotonergic agent. The resulting average increase in time to the first ejaculation is given in the table below.

| | | Potential average |
|---|---|---|
| 5-HT1A-receptor | | increase in time to |
| antagonist | Compound class or use | first ejaculation (%) |
| Alprenolol | Non-selective beta blocker | 75 |
| Cyproheptadine | Antihistamine | 78 |
| Dotarizine | Calcium channel blocker | 67 |
| DU125530 | | 254 |
| Methiothepin | Antipsychotic | 97 |
| Nebiovolol | Beta blocker | 95 |
| Pindolol | Selective beta blocker | 46 |
| Propranolol | Beta blocker | 87 |
| Risperidone | Antipsychotic | 102 |
| Spiperone | Anti-schizophrenic | 80 |

### REFERENCES

Bijlsma E Y, Chan J S W, Olivier B, Veening J G, Millan M J, Waldinger M D, Oosting R S. Sexual side effects of serotonergic antidepressants: Mediated by inhibition of serotonin on central dopamine release? Pharmacol Biochem Behav 121: 88-101, 2014.
Chan J S W, Waldinger M D, Olivier B, Oosting R S. Drug-induced sexual dysfunction in rats. Current protocols in Neuroscience Suppl 53: 9.34.1-9.34.11, 2010.
Chan J S W, Snoeren E M S, Cuppen E, Waldinger M D, Olivier B, Oosting R S. The serotonin transporter plays an important role in male sexual behavior: a study in serotonin transporter knockout rats. J Sex Medicine 8:97-108, 2011.
Joordens R J E, Hijzen T H, Olivier B. The effects of 5-HT1A receptor agonists, 5-HT1A receptor antagonists and their interaction in the fear-potentiated startle response paradigm. Psychopharmacology 1998; 139: 383-390.
Mos J, Van Hest A, Herremans A H J, Van Drimmelen M, Olivier B. The putative 5-HT1A receptor antagonist DU125530 blocks the discriminative stimulus effects of the 5-HT1A agonist flesinoxan in the pigeon. Eur. J. Pharmacol. 1997; 325: 145-153.
Olivier B, Chan J S W, Pattij T, de Jong T R, Oosting R S, Veening J G, Waldinger M D. Psychopharmacology of male rat sexual behavior: modeling human sexual dysfunctions? International Journal of Impotence Research 18: S 14-S23, 2006.
Olivier J D A, Esquivel Franco D C, Waldinger M D, Olivier B. Sexual dysfunction, depression and antidepressants: a translational approach. In: Sexual Dysfunction, Intech open, Ed. B. Olivier, pp 59-76, 2017.
RABINER E A, WILKINS M R, TURKHEIMER, GUNN R N, UDO DE HAES J, DE VRIES M, GRASBY P M. 5-Hydroxytryptamine1A Receptor Occupancy by Novel Full Antagonist 2-[4-[4-(7-Chloro-2,3-dihydro-1,4 benzdioxyn-5-yl)-1-piperazinyl]butyl]-1,2-benzisothiazol-3-(2H) one-1,1-dioxide: A [11C][O-methyl-3H]—N-(2-(4-(2-methoxyphenyl)-1-piperazinyl)ethyl)-N-(2-pyridinyl)cyclohexanecarboxamide Trihydrochloride (WAY-100635) Positron Emission Tomography Study in Humans. J Pharmacol Exp Ther 301:1144-1150, 2002.

1.-8. (canceled)

9. A method of preventing and/or treating premature ejaculation in a subject, the method comprising:
administering to the subject at least one compound of Formula I 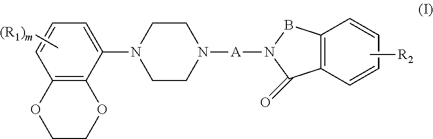
wherein
R₁ is a halogen, methyl, methoxy, hydroxyl, trifluoromethyl or cyano,
m is 1 or 2,
R₂ is hydrogen or chlorine,
A represents an alkylene chain containing 2-6 C-atoms, which may be substituted with a phenyl group, and
B is methylene, ethylene, carbonyl, sulfinyl, sulfonyl, or sulfur, or a salt thereof in combination with at least one serotonergic agent or a salt thereof,
wherein the at least one compound is administered separately, sequentially, or simultaneously with the at least one serotonergic agent.

10. The method according to claim 9, wherein at least one compound of Formula I is 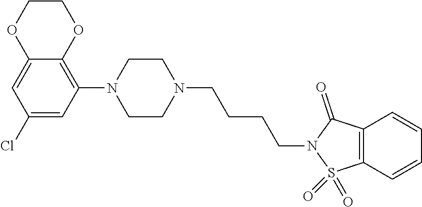 [SMILES: O=C1c2ccccc2S(=O)(=O)N1CCCCN1CCN(c2cc(Cl)cc3c2OCCO3)CC1]
or a salt thereof.

11. The method according to claim 9, wherein administration of the compound and the at least one serotonergic agent causes the subject to experience increased latency time to ejaculation.

12. The method according to claim 10, wherein administration of the compound and the at least one serotonergic agent causes the subject to experience increased latency time to ejaculation.

13. The method according to claim 9, wherein
the at least one compound is administered between 0.5-1.5 hours before sexual activity of the subject; and/or
the at least one serotonergic agent is administered between 0.5-1.5 hours before sexual activity of the subject.

14. The method according to claim 13, wherein peak pharmacological effects in the subject of the at least one compound and/or the at least one serotonergic agent partly overlap.

15. The method according to claim 9, wherein the at least one compound and/or the at least one serotonergic agent are provided as a composition comprising the at least one compound and/or the at least one serotonergic agent, and optionally further comprising one or more pharmaceutically acceptable carriers, excipients, and/or diluents.

16. The method according to claim 9, wherein the at least one compound and/or the at least one serotonergic agent are provided as a kit of parts comprising the at least one compound and/or the at least one serotonergic agent.

17. The method according to claim 9, wherein administration of the at least one compound and/or the at least one serotonergic agent is oral.

18. The method according to claim 9, wherein administration of the at least one compound and/or the at least one serotonergic agent is sublingual.

19. The method according to claim 9, wherein the at least one serotonergic agent is at least one, two, or three agents selected from the group consisting of a serotonin receptor agonist or antagonist, a serotonin reuptake inhibitor, a selective serotonin and noradrenalin reuptake inhibitor, and a serotonin releasing agent.

20. The method according to claim 9, wherein the at least one serotonergic agent is a selective serotonin reuptake inhibitor.

21. The method according to claim 20, wherein the selective serotonin reuptake inhibitor is selected from the group consisting of citalopram, escitalopram, fluoxetine, fluvoxamine, paroxetine, sertraline, dapoxetine, indalpine, zimelidine, alaproclate, centpropazine, cericlamine (JO-1017), femoxetine (Malexil®; FG-4963), ifoxetine (CGP-15210), omiloxetine, panuramine (WY-26002), pirandamine (AY-23713), and seproxetine ((s)-norfluoxetine).

22. A method of increasing latency time to ejaculation in a male subject, the method comprising:
administering to the subject, before sexual activity, a compound: 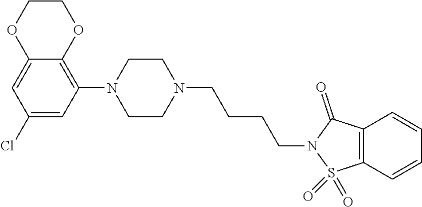 [SMILES: O=C1c2ccccc2S(=O)(=O)N1CCCCN1CCN(c2cc(Cl)cc3c2OCCO3)CC1]
or a salt thereof in combination with
at least one serotonergic agent or a salt thereof,
wherein the at least one compound of Formula I is administered separately, sequentially, or simultaneously with the at least one serotonergic agent,
so that the subject experiences increased latency time to ejaculation.

23. The method according to claim 22, wherein the at least one serotonergic agent is at least one, two, or three agents selected from the group consisting of a serotonin receptor agonist or antagonist, a serotonin reuptake inhibitor, a selective serotonin and noradrenalin reuptake inhibitor, and a serotonin releasing agent.

24. The method according to claim 22, wherein the at least one serotonergic agent is a selective serotonin reuptake inhibitor.

25. The method according to claim 24, wherein the selective serotonin reuptake inhibitor is selected from the group consisting of citalopram, escitalopram, fluoxetine, fluvoxamine, paroxetine, sertraline, dapoxetine, indalpine, zimelidine, alaproclate, centpropazine, cericlamine (JO-1017), femoxetine (Malexil®; FG-4963), ifoxetine (CGP-15210), omiloxetine, panuramine (WY-26002), pirandamine (AY-23713), and seproxetine ((s)-norfluoxetine).
